(19) United States
(12) Patent Application Publication
(10) Pub. No.: US 2003/0068613 A1
(43) Pub. Date: Apr. 10, 2003

(54) Method for studying regeneration in biological material

(76) Inventors: Cornelia Leibrock, Edingen (DE); Thomas Muller, Wuppertal (DE); Hansjurgen Volkmer, Tubingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/234,728

(22) Filed: Aug. 30, 2002

Related U.S. Application Data
(63) Continuation of application No. PCT/EP01/02332, filed on Mar. 1, 2001.

(30) Foreign Application Priority Data
Mar. 1, 2000 (DE) 100 09 722.7

(51) Int. Cl. C12Q 1/00; C12Q 1/68; G01N 33/53; G01N 33/567

(52) U.S. Cl. 435/4; 435/6; 435/7.2; 205/777.5

(57) ABSTRACT

In a method for studying regeneration in biological material said biological material is cultivated on an electrode array, subjected to the action of regeneration promoting active substances and/or physical processes, whereupon the regeneration process of said biological material is analyzed.

### RELATED APPLICATIONS

This is a continuation of International Application PCT/EP01/02332, with an international filing date of Mar. 1, 2001, published in German under PCT Article 21(2) and now abandoned, which claims priority to a German application No. 10009722.7, filed Mar. 1, 2000.

### BACKGROUND OF THE INVENTION

1. Field of the Invention

The present invention is concerned with systems for studying or investigating regeneration, synaptogenesis and synaptic stability in biological material, for example cells.

The invention is further concerned with injuries to the central nervous system (CNS) of adult mammals caused, for example, by trauma, ischaemia or neurodegenerative diseases. These injuries result in serious and lasting functional deficits which necessitate treatment.

2. Related Prior Art

It is already known that, in contrast to the developing CNS or to peripheral nerves, the CNS axons at a certain point in their development lose their ability to regenerate after axotomy. It is assumed that this loss is due to the non-permissive environment of the CNS and to factors intrinsic to the adult CNS neurons.

Treatments which can lead to neural regeneration include the administration of active substances, for example drugs, or the use of physical processes, for example electrostimulation.

No reliable method is presently known with which it is possible for these treatments, which can lead to neuronal regeneration, to be evaluated in the context of a model system which correlates long-term recordings of physiological activity with morphological changes.

Li et al. in Entorhinal Axons Project to Dentate Gyrus in Organotypic Slice Co-culture, NEUROSCIENCE 1993, volume 52, pages 799-813, describe an organotypic co-culture of entorhinal cortex and postnatal hippocampus which has been freed from its own entorhinal access. By immunohistochemistry, the authors demonstrate in this in vitro system the formation of entorhinal projections in the co-cultured hippocampus, for which purpose the slices were stained and evaluated under a microscope. The authors also show a small number of electrophysiological data which were obtained with a stimulation electrode of tungsten and a recording electrode of glass.

The measurement method described here is not suitable as a model system for long-term recordings.

In a further publication, Li et al. describe, at the anatomical level, the exclusivity of the connections between entorhinal cortex and dentate gyrus in the publication: Connectional Specification of Regenerating Entorhinal Projection Neuron Classes Cannot Be Overriden by Altered Target Availability in Postnatal Organotypic Slice Co-culture, EXPERIMENTAL NEUROLOGY, 1996, volume 142, pages 151-160.

Baker et al. in Chronic Blockade of Glutamate-mediated Bioelectric Activity in Long-term Organotypic Neocortical Explants Differentially Effects Pyramidal/Non-pyramidal Dendritic Morphology, DEVELOPMENTAL BRAIN RESEARCH, 1997, volume 104, pages 31-39, show regeneration in another organotypic preparation, namely in cortex-cortex co-cultures. Here too, electrophysiological data were obtained using glass micro-electrodes.

Nessler and Mass, in Direct-current Electrical Stimulation of Tendon Healing in vitro, CLINICAL ORTHOPAEDICS, 1987, volume 217, pages 303-312, report on the possibility of stimulating the regeneration of tendons in vitro using direct current.

In addition, Borgens, in Electrically Mediated Regeneration and Guidance of Adult Mammalian Spinal Axons into Polymeric Channels, NEUROSCIENCE, 1999, volume 91, pages 251-264, was able to show that an extracellular electrical field promotes the regeneration of nerve fibres in the adult mammalian spinal cord.

The prior art thus far described therefore reveals principal model systems which are suitable for investigating regeneration in biological material; reliable long-term measurements of physiological activity and its correlation with morphological changes cannot however be made using the measurement method described in the prior art.

WO 00/79273 A2, published only after the priority date of the present application, describes a method in which hippocampus tissue is arranged on a micro-electrode array via which the nerve tissue is stimulated and the different reactions to different psychopharmaceuticals are measured.

### SUMMARY OF THE INVENTION

Against this background, it is an object of the present invention to provide a method for studying treatments, which can lead to neural regeneration, in a model system which correlates long-term recordings of physiological activity with morphological changes.

According to the invention, this object is achieved by using an electrode array in order to evaluate or investigate, in biological material, a regeneration which is promoted by active substances or physical processes, the electrode arrangement used preferably being a micro-electrode arrangement.

The inventors in the present application have found that in this context, in particular, organotypic cultures and co-cultures of tissue from the central nervous system can be used to functionally measure the development and regeneration of connections between cells and to investigate the action of drug administration or physical processes on the activation of cell functions.

Promoting regeneration is to be understood, in the context of this application, both as initiating and also as supporting the regeneration.

The inventors have also found and demonstrated that electrode arrays generally, in particular micro-electrode arrays, which are described for example by Egert et al. in A Novel Organotypic Long-term Culture of the Rat Hippocampus on Substrate Integrated Multielectrode Arrays, BRAIN RESEARCH PROTOCOLS, 1998, volume 2, pages 229-242, are particularly well suited for longterm measurement in such systems.

By combination of organotypic co-cultures with extracellular micro-electrode recording technology, the development of new connections and their regenerative ability at a functional level can be repeatedly monitored for days or weeks in the same culture.

In this context, organotypic cultures represent a good alternative to animal experiments for tackling questions in the area of regeneration. The co-culture model of dentate gyrus and entorhinal cortex affords a favorable starting point. From the literature described at the outset, it can be taken that this co-culture model in the literature is morphologically well characterized, and the analogy to the in vivo situation has been demonstrated. Very little electrophysiological data is available from the literature, however, but these data can now be made available by means of the use according to the invention of the micro-electrode array (hereinafter the MEA). A further advantage is that between the two tissues there is a single monosynaptic connection with clearly defined starting point and end point.

Between the dentate gyrus and the entorhinal cortex there is a connection called perforant pathway which has been described in detail in the literature and which is in part represented by this monosynaptic connection.

The inventors in the present application have now found, for the first time, that by using micro-electrode arrays it is possible to analyze the generation of a previously experimentally interrupted perforant pathway both during the juvenile phase, when regeneration is still relatively easily possible even in the central nervous system, and also in the differentiated state. The use of MEA technology in conjunction with the organotypic culture mentioned by way of example affords the possibility of repeatedly determining electro-physiologically the activity across the whole area of a preparation over a period of days and weeks. Electrostimulation at reproducible and always identical sites with simultaneous recording in the whole of the remaining co-culture is also possible in order to demonstrate a connection between both explants.

The initial connection of the explants takes place in the juvenile phase. If a lesion is then made through the newly developed connection, after the co-culture has reached a differentiated state, the regeneration can also be analyzed in the adult-like state.

In this kind of long-term monitoring of 17 recorded co-cultures on an MEA with electrodes which had a spacing of 200 μm and a diameter of 30 μm, it was possible to show that twelve of these co-cultures had again grown together. Seven of these co-cultures which had grown together were lesioned, and four of these were still vital after the lesion.

In one instant it was possible to show that, using FK506, an immunosuppressant, a co-culture regenerated the perforant pathway.

This not only represents a test for the regenerative potential of the differentiated neurons; rather, it is possible also to test the regenerative potential of drugs and of physical processes for activating cell functions, for example by functional electrical stimulation. This can be effected, for example, by direct electrical stimulation or by applying an electrical or electromagnetic field, which can likewise be done with the aid of an MEA.

According to the invention, the drugs or active substances are chosen from the group consisting of: organic or chemical compounds and biologically active substances, for example peptides, proteins, nucleic acids, and the biological material can include both juvenile cells and also cells in the adult-like state.

By means of the use according to the invention, it is now possible to analyze the regeneration of intercellular connections, the functional connection or reconnection between neurons, a time course of developing links or reconnections, and the regeneration of cells that have been damaged regarding their intercellular links.

According to the invention, the regeneration can also be observed repeatedly over a relatively long period of time, preferably of more than one week, on cultures and co-cultures. It is moreover possible to analyze the reactions of receptor systems to the application of active substances.

Against this background, another object of the invention is a method for analyzing the effects of drugs on the regeneration potential of functional connections in cultures, in which method an electrode array, preferably as described above, is used.

Here, it is first necessary to verify pharmacologically which receptor systems in the co-culture are important, for example, for connecting entorhinal cortex to the dentate gyrus. For this purpose, the action of antagonists of the various potentially relevant receptors on the activity in connected co-cultures must be tested.

It goes without saying that the aforementioned features, and the features still to be discussed below, can be used not only in the respectively cited combination, but also in other combinations or in isolation, without departing from the scope of the present invention.

### BRIEF DESCRIPTION OF THE DRAWINGS

Further advantages will become evident from the following illustrative embodiments and in conjunction with the drawing, in which:

FIG. 1 shows a diagrammatic representation of the in vivo situation between entorhinal cortex (ec) and dentate gyrus (dg);

FIG. 2 shows the preparation of co-cultures on a micro-electrode array;

FIG. 3 shows the electrical stimulation on co-cultures as in FIG. 2;

FIG. 4 shows the restoration of functional connections in a co-culture as in FIG. 2; and

FIG. 5 shows the electrical stimulation of electrogenic cells in the cell aggregate on an MEA.

### DETAILED DESCRIPTION OF PREFERRED EMBODIMENTS

### Example 1

Measurements on a Co-culture

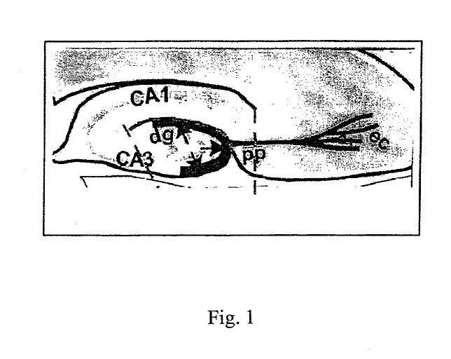

FIG. 1 is a diagrammatic illustration of the in vivo situation between entorhinal cortex (ec) and dentate gyrus (dg). The indication pp shows a part of the perforant pathway which, during the preparation, is sectioned along the broken line shown. CA1 and CA3 indicate fields of the hippocampus.

Sections of entorhinal cortex and dentate gyrus which were taken from 6-day-old to 7-day-old Wistar rats or BalbC mice were cultured on a micro-electrode array of 60 substrate-integrated electrodes. The electrodes had a spacing of 200 μm and a diameter of 30 μm. The slices were stimulated and the electrophysiological activity recorded using an MCS amplifier and a data acquisition system (Multi Channel System, Germany), which permits the use of each contact as stimulation or recording electrode without manipulation of the slices. The data of all 60 channels were recorded online at a scanning frequency of 25 kHz/channel.

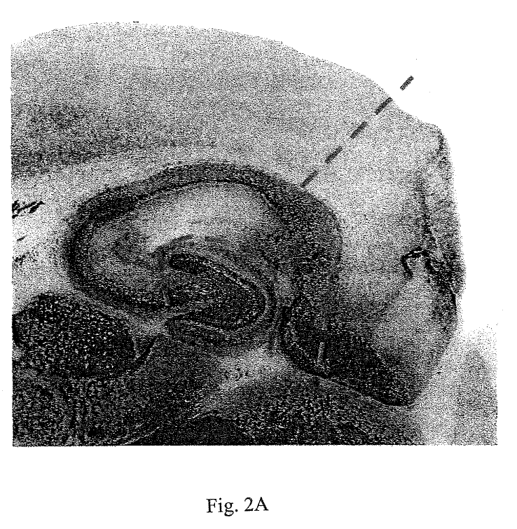

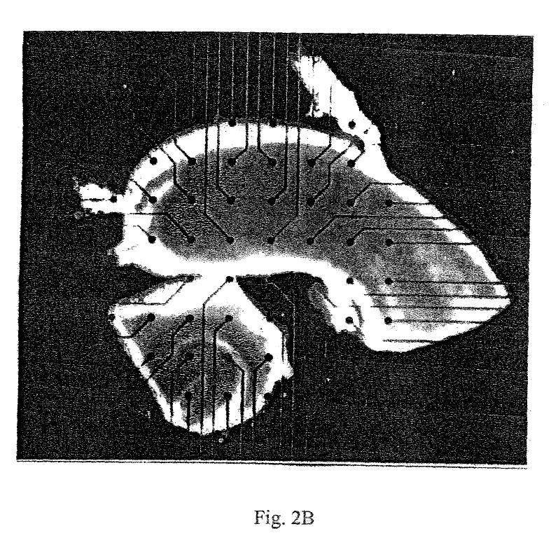

FIG. 2 shows one such preparation of co-cultures for which horizontal brain slices (425 μm) were used. The entorhinal cortex part and the corresponding dentate gyrus which represent the origin and the target tissue of part of the perforant pathway were separated from the brain slices. The cuts made during the preparation are shown by broken lines in FIG. 2a.

The two explants were then positioned, similarly to the in vivo situation, on a micro-electrode array with an 8×8 field of extracellular electrodes. FIG. 2b shows a co-culture immediately after preparation.

Starting from the seventh day in vitro (DIV7), the overall spontaneous activity of the co-cultures and the reaction to extracellular electrical stimulation of layer II of the entorhinal cortex and of the stratum granulosum of the dentate gyrus were monitored every three days.

In addition, Dil staining (1,1-dioctadecyl-3,3,3,3-tetramethylindocarbocyanine perchlorate; Honig and Hume, Fluorescent Carbocyanine Dyes Allow Living Neurons of Identified Origin to be Studied in Long-term Cultures, J. CELL. BIOL. 1986, volume 103, pages 171-187) of the dg gave retrograde labelling of the cells in layer II of the ec.

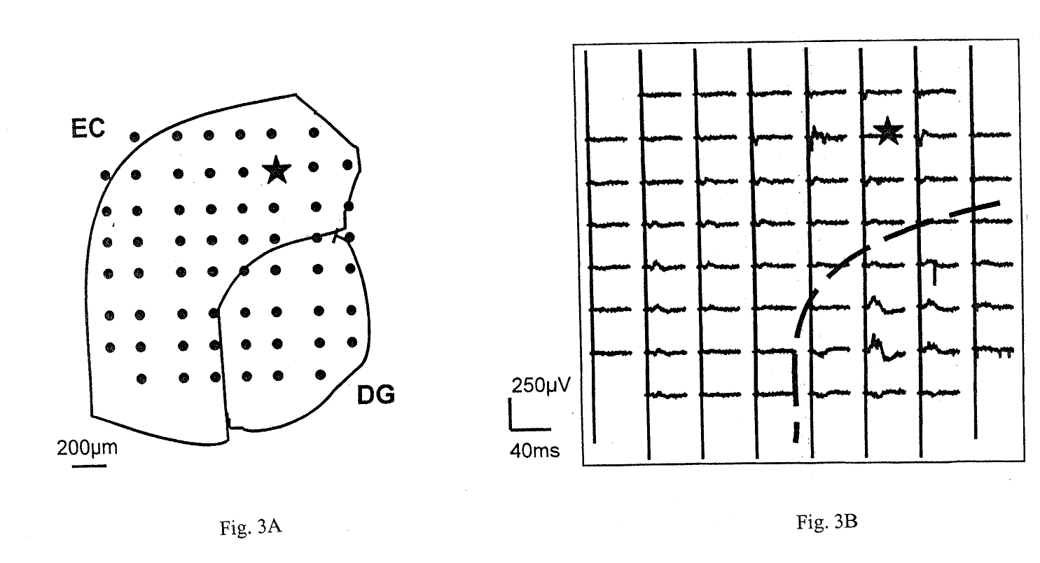

FIG. 3 shows the extracellular stimulation of a co-culture of ec and dg, where FIG. 3a indicates the morphology of the co-culture after 7 days in vitro (DIV7). FIG. 3b shows the reaction on all MEA electrodes after electrical stimulation in layer II-III of the ec (80 μA, 150 μs).

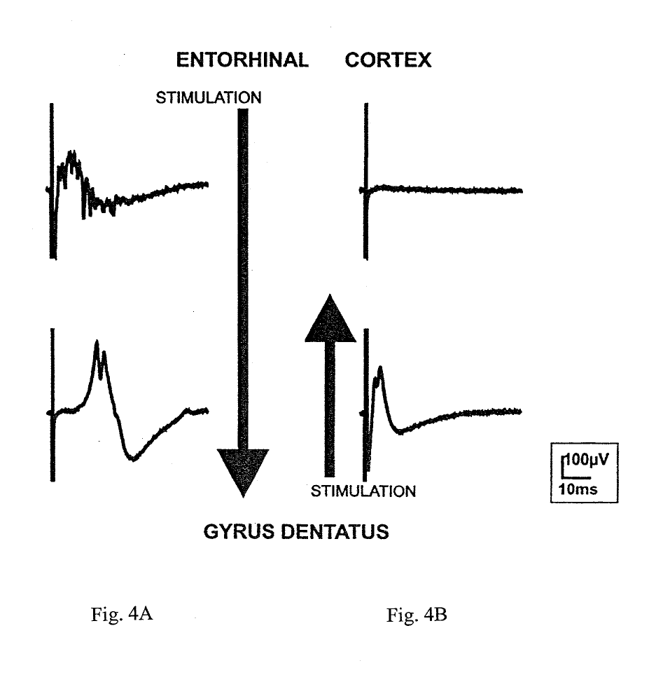

FIG. 4 shows the functional restoration of connections in cocultured explants of dg and ec from juvenile rats or mice. After 7 days in vitro, it is possible to incite activity in the dg via electrical stimulation in the ec (FIG. 4a), but not vice versa (FIG. 4b). This corresponds with the in vivo situation in which ec layer II cells project into the dg. Of 112 cultures investigated, 85 showed this one-way connection, 20 showed a two-way connection, and 7 showed no connection at all.

From these results it is possible to conclude that, independently of the loss of the intermediate subicular tissue, the axons of the entorhinal cortex form correct and functional connections with the dentate gyrus which are similar to the perforant pathway known in vivo. It is unlikely that the connections observed derive from unspecified axonal sprouting, because the dg axons also show extensive projections from the explant but do not produce any functional connections with the cortex.

The linked co-culture thus established now develops in vitro to an adult-like stage, at which point lesions are made in the newly developed connections.

With these lesions it is then possible to test the regenerative potential of the differentiated neurons and the promotion, that is to say initiation or support, of the regeneration by drugs or physical processes.

### EXAMPLE 2

Investigations at Different Stages of Regeneration

Axonal Growth

Neuroproductive or regeneration-promoting substances which act directly on the starting cells in layer II of the ec can permit a regeneration of the pathway already established once in vitro, which is not normally possible in the differentiated state.

Here, the experimental possibility exists of applying FK506, for example, into the culture medium after lesion. In this context, it is important that the culture has reached a sufficient age in order to rule out development-related growth of the neurites. This is the case after DIV7 or later.

Target-seeking

It is highly probable that the repulse action of semaphorin III mediated by the neuropilin I receptor contributes to the correct, targeted connection of dg and ec.

It is experimentally possible, with peptides which cross membranes, to inhibit the signal cascade in question and to suppress the semaphorin III action. The peptides are added to the culture medium directly after preparation, so that a lesion is not required.

Synaptogenesis

It has been found that the interaction with intracellular proteins is essential for the correct arrangement of the receptors and other membrane proteins in the synapse. Many of these intracellular proteins share a common motive, the so-called PDZ binding site, with which they bind to the membrane proteins. The functional relevance of these interactions is still largely unknown, but it can be investigated with the measurement method described here.

Here too, the interaction of the receptors with the PDZ proteins can be inhibited by PDZ antagonists which cross the membrane; this can take place during re-growth or thereafter. This affords the possibility of comparing the situation before and after on one slice (internal control).

### EXAMPLE 3

Immunohistochemical and Electro-physiological Investigation

Organotypic cortical co-cultures (350-425 μm thick from the neocortex) were prepared from three-day-old to seven-day-old Wistar rats of both sexes and were maintained in vitro using the roller tube technique; on this point see Gähwiler, Organotypic Monolayer Cultures of Nervous Tissue, J. NEUROSCIENCE METHODS, 1981, volume 4, pages 329-342. The co-cultures were allowed to grow for up to 35 days either on glass cover plates or on micro-electrode arrays in medium which contained 50% Eagle's basic medium, 25% Hanks-buffered saline solution, 25% horse serum, 33 mM D-glucose and 1 mM L-glutamine. The medium was replaced twice a week, and the co-culture explants were positioned relative to one another with varied orientations and varied distances (0 to 500 μm).

The explants cultured on glass cover plates were fixed in buffered 4% paraformaldehyde for two hours, washed, treated in buffered 0.1% Triton X-100, washed again, and pre-incubated in 1% bovine serum albumin (BSA) and 0.1% goat serum, in order to prevent non-specific binding.

The cultures were incubated for 3 to 9 days with primary antibody in 1% buffered BSA solution. After four to five days of washing in buffer, the cultures were incubated with a secondary antibody (goat anti-mouse IgG, CY3) for three to five days and afterwards washed for four to five days. The explants were placed on slides, viewed by fluorescence and photographed. The controls consisted of explants which were incubated only with the secondary antibody. Primary antisera (Boehringer, Mannheim) were monoclonal antibodies which were diluted to working solutions of 0.5 μg/ml and 1 μg/ml for anti-GAP-43 and synapto-physin, respectively.

Nissl stains were used to assess the morphology of the explants. The co-cultures were fixed, dehydrated, dyed in toluidine blue, placed on slides, and photographed.

The explants cultured on micro-electrode arrays were recorded at different times for spontaneous electrical activity, at the earliest after 4 DIV (days in vitro) and at the latest 35 DIV in normal culture medium at a temperature of 35° C. Using a multi-channel recording system, the 60 micro-electrodes could be recorded simultaneously, as a result of which it was possible to test correlated activity in the co-cultures. This revealed the restoration of functional connections between the explants. The electrodes had impedances of 100 to 300 kΩ (at 1 kHz), a spacing of 500 μm and a diameter of 10 μ. The activity was recorded at 10 to 25 kHz per channel, stored, and analyzed offline in respect of latency.

While the explants were perfused with carbogen-equilibriated ACF, consisting of (in each case in ml/l) 125 NaCl, 3.5 KCl, 1.3 MgSO₄, 1.2 KH₂PO₄, 2.4 CaCl₂, 26 NaHCO₃, 10 glucose (ph 7.4), at a flow rate of approximately 0.5 ml/min, the neurosensitivity to various channel blockers and receptor agonists and antagonists was tested. The explants were discarded after the pharmacologoical experiments.

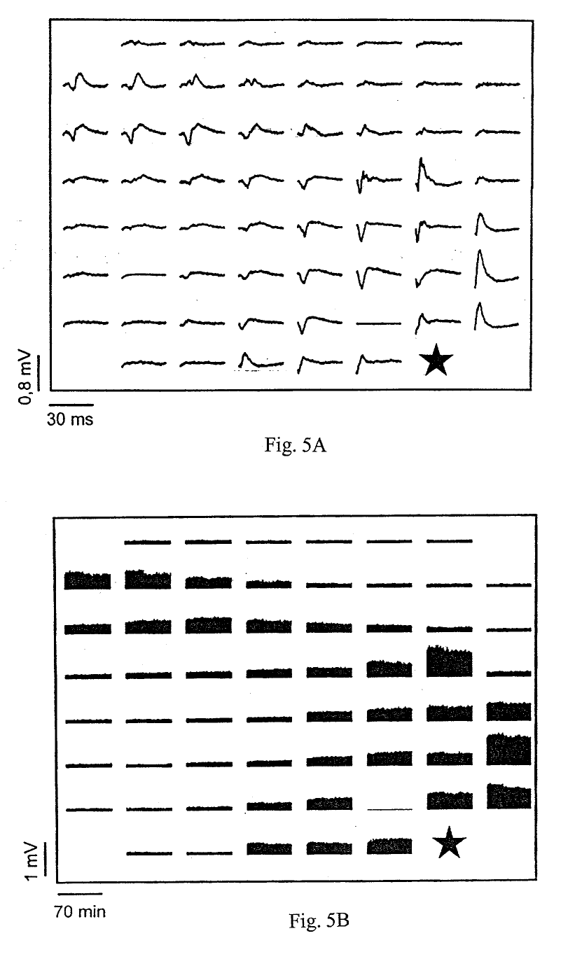

FIG. 5 shows an electrical stimulation of electrogenic cells in the cell aggregate on a micro-electrode array, as was used in the tests described here. FIG. 5a shows simple responses in an organotypic hippocampus culture after 17 days in vitro with monopolar electrical stimulation via the MEA electrode marked with a star. Stimulation artefacts are not shown.

FIG. 5b shows a long-term stimulation over more than one hour, in which uniform responses were obtained on the various electrodes. Each column shows the amplitude of the response to stimuli applied every 60 seconds in the electrode marked with a star.

### EXAMPLE 4

Tests of FK506

FK506, an immunosuppressant which has been shown to have neuro-protective and neurogenerative effects in the central and peripheral nervous system (Brecht and Herdegen, 1999, Der neue “Dreh”: Hemmung von FKBP-Rotamasen als neurogeneratives und europrotektives Prinzip, Neuroform 5:36-43), was added to the culture medium at 0 DIV in a concentration of 50 nM and again administered with each change of the culture medium up to 14 DIV. The co-cultures were tested for correlated activity, and the percentage of the explants showing correlation within a whole group of the treated explants was compared with controls in which no explant was treated with the active substance.

In the untreated control (n=28), a correlated activity was found in ca. 60% after 14 days in vitro, whereas in the explants treated with FK506 (n=18) a correlated activity of almost 90% was found. The expression “correlated activity” is to be understood in this context as meaning the regeneration of functional connections between the sections.

What is claimed is:

1. A method for studying regeneration in a biological material comprising:
cultivating said biological material on an electrode array;
subjecting said biological material to a regeneration-promoting process; and
analyzing effectiveness of said regeneration-promoting process in said biological material by measuring electrical activity of said biological material through said electrode array.

2. The method of claim 1, wherein said electrode array is a microelectrode array.

3. The method of claim 1, wherein said regeneration-promoting process comprises administering a chemical substance.

4. The method of claim 1, wherein said regeneration-promoting process comprises subjecting said biological material to a physical process.

5. The method of claim 3, wherein said chemical substance is selected from the group consisting of: drugs, peptides, proteins and nucleic acids.

6. The method of claim 4, wherein said physical process is electrical stimulation, electrical field, or electromagnetic field.

7. The method of claim 1, wherein said regeneration is regeneration of intercellular connections.

8. The method of claim 1, wherein said regeneration is regeneration of functional connections between neurons.

9. The method of claim 7, wherein a time course of said regeneration of intercellular connections is analyzed.

10. The method of claim 8, wherein a time course of said regeneration of functional connections between neurons is analyzed.

11. The method of claim 1, wherein the regeneration is repeatedly observed for at least a week.

12. The method of claim 1, wherein said biological material comprises juvenile cells.

13. The method of claim 1, wherein said biological material comprises cells in an adult state.

14. The method of claim 3, wherein reactions of receptor systems to application of said chemical substance are analyzed.

15. The method of claim 8, wherein said regeneration of functional connections between neurons is studied in the presence of a drug.
